(19) United States
(12) Patent Application Publication
(10) Pub. No.: US 2015/0284715 A1
(43) Pub. Date: Oct. 8, 2015

(54) Enrichment Methods

(71) Applicant: QIAGEN GmbH

(72) Inventors: Matthias Wahl, Neuss (DE); Nan Fang, Neuss (DE); Kerstin Goebels, Laichlingen (DE)

(21) Appl. No.: 14/277,818

(22) Filed: May 15, 2014

Related U.S. Application Data
(60) Provisional application No. 61/976,017, filed on Apr. 7, 2014.

(51) Int. Cl. C12N 15/10

(52) U.S. Cl. CPC C12N 15/1068

(57) ABSTRACT

Methods are described for the separation of microspheres covered with nucleic acids of interest from undesired microspheres and/or molecules. These separations may be negatively affected by the presence of non-specific interactions between nucleic acids or microspheres.

### FIELD OF INVENTION

Methods and compositions for improving the enrichment of a population of particles containing an analyte are disclosed. The technique finds many uses, including enriching for beads with clonally amplified template, which can be used in a variety of assays, including nucleic acid sequencing.

### BACKGROUND

Next generation sequencing (NGS), or massively parallel sequencing, where millions to hundreds of millions of reads can be generated in the same sequencing run, is a new technology that has already found numerous applications in research and clinical areas. All next generation sequencing methods require prior clonal amplification of nucleic acid fragments before sequencing. To achieve this, most NGS platforms from major suppliers (with the exception of Illumina) employ microsphere-based clonal amplification of nucleic acids by polymerase chain reaction (PCR).

To achieve that single library molecules are amplified on single microspheres, microemulsions are generated (emulsion PCR) which statistically contain one bead and less than one library molecule per droplet (thereby ensuring that no droplet contains two library molecules). As a consequence, several microspheres lack amplicon (hereafter called ‘null beads’) after emulsion PCR. To ensure a high throughput of the succeeding NGS sequencing reaction, these null beads are therefore depleted by a process called ‘enrichment’ where amplicon-containing microspheres (hereafter called ‘live beads’) are affinity purified.

What is needed are methods to improve enrichment so that higher numbers of live beads are recovered.

### SUMMARY OF THE INVENTION

Methods and compositions for improving the enrichment of a population of particles containing an analyte are disclosed. The technique finds many uses, including enriching for beads with clonally amplified template, which can be used in a variety of assays, including nucleic acid sequencing.

Microspheres are a commonly used tool for nucleic-acid based applications in the fields of basic biological research, biomedical research, applied testing, and molecular diagnostics. Applications include, but are not limited to, clonal amplification of specific DNA fragments on the surface of microspheres by polymerase chain reaction or other amplification methods, and specific isolation of nucleic acids/nucleic acid with oligo-conjugated microspheres by hybridization-based methods. A critical step for above applications is the separation of microspheres covered with nucleic acids of interest from undesired microspheres and/or molecules. These separations may be negatively affected by the presence of non-specific interactions between nucleic acids or microspheres.

Here, we describe a novel method capable of reducing non-specific interactions during the microsphere-based isolation of nucleic acids and/or nucleic acid covered microspheres, thereby improving the efficiency and effectiveness of the respective methods. The method utilizes an enzymatic reaction to specifically degrade non-target nucleic acids that can lead to unspecific binding to capture microspheres while leaving the target nucleic acid intact, thereby enhancing the efficiency and specificity of the capture of the target nucleic acids, or microspheres containing target nucleic acids,

One specific application of the invention is to increase the efficiency of the enrichment of amplicon-covered microspheres (hereafter called ‘live beads’) from non-amplicon covered microspheres (hereafter called ‘null beads’) in NGS applications. In one embodiment, the live/null bead mixture is pre-treated with a nuclease, including but not limited to, an endonuclease or an exonuclease. In one embodiment, the present invention contemplates use of an exonuclease that catalyzes the removal of nucleotides from single-stranded DNA in the 3′ to 5′ direction (e.g. E. coli Exonuclease I) prior to enriching biotinylated live beads by streptavidin-coated microspheres (hereafter called “capture beads” or “enrichment beads”). In one embodiment, the single-strand specific nuclease is selected from the group consisting of Si nuclease, Mung Bean Nuclease, BAL 31 nuclease.

In one embodiment, the present invention contemplates a method of recovering amplified nucleic acid, comprising: a) providing i) a plurality of amplification beads, amplification reagents, a first primer (e.g. in solution or immobilized on said beads), a second primer (e.g. preferably in solution when the first primer is immobilized on the beads), and template; ii) enrichment beads, wherein said enrichment beads are different from said amplification beads, and iii) a single-strand specific nuclease; b) exposing said amplification beads to conditions so as to amplify at least some of said template on at least some of said beads so as to create processed beads; c) treating said processed beads with said single-strand specific nuclease so as to create treated beads; and d) contacting said treated beads with said enrichment beads, wherein said treated beads comprising amplified template bind to said enrichment beads so as to make a population of bead complexes, thereby recovering amplified nucleic acid.

In one embodiment, said treated beads not comprising amplified template do not bind to said enrichment beads in step d). In one embodiment, a portion of said treated beads of step d) comprise amplicon labeled with biotin and said enrichment beads comprise streptavidin-coated microspheres. In one embodiment, biotin is introduced into said amplicon during amplification of step b) so as to create said amplicon labeled with biotin. In another embodiment, biotin-labeled oligonucleotides were hybridized to said amplicon after step c) so as to create said amplicon labeled with biotin. In one embodiment said amplification reagents comprise PCR reagents.

In one embodiment, the present invention contemplates a method of enriching, comprising: a) providing i) an emulsion comprising one or more aqueous compartments in oil, at least some of said compartments comprising PCR reagents, a first primer immobilized on an emulsion bead, a second primer in solution, and template; ii) enrichment beads, wherein said enrichment beads are different from said emulsion beads in said compartments, and iii) a single-strand specific nuclease; b) exposing said emulsion to conditions so as to amplify at least some of said template on at least some of said emulsion beads in at least some of said compartments; c) breaking said emulsion under conditions such that said emulsion beads are recovered; d) treating said recovered emulsion beads with said single-strand specific nuclease to as to create treated beads; and e) enriching for treated beads comprising amplified template by contacting said treated beads with said enrichment beads, wherein said treated beads comprising amplified template bind to said enrichment beads so as to make a population of treated bead—enrichment bead complexes. In a preferred embodiment, emulsion beads not comprising amplified template do not bind to said enrichment beads in step e).

It is not intended that the present invention be limited by the nature of the emulsion beads. Beads of various types can be used. In one embodiment, said emulsion beads are magnetic.

It is not intended that the present invention be limited by the method by which the emulsion is broken. In one embodiment, the emulsion is broken using isopropanol.

In one embodiment, the method further comprises f) capturing at least some of said population of complexes under conditions such that a majority of said emulsion beads not comprising amplified template are not captured. In one embodiment, the capturing in step f) comprises size selection. In one embodiment, said size selection comprises density centrifugation. In one embodiment, said size selection comprises capturing at least some of said population of complexes on a surface. In one embodiment, said surface comprises the surface of a filter. In one embodiment, said filter is a single layer nylon mesh. In one embodiment, said filter is positioned in a spin column. In one embodiment, said spin column is centrifuged during step f) so as to facilitate passage of said uncaptured emulsion beads through said filter.

In one embodiment, said enrichment beads are different in size from said emulsion beads. In one embodiment, said enrichment beads are at least five times and up to one hundred times larger than said emulsion beads.

In one embodiment, the method further comprises, after step f): g) subjecting said population of complexes to conditions so as to separate said emulsion beads comprising amplified template from said enrichment beads such that the majority of said emulsion beads comprising amplified template separate from said enrichment beads. It is not intended that the present invention be limited to any specific condition for separating live beads from the enrichment beads. In one embodiment, denaturing conditions are used. In one embodiment, NaOH denaturation is used for separation. In one embodiment, said emulsion beads are magnetic and said emulsion beads (once separated from said enrichment beads) are exposed to a magnet.

In one embodiment, the emulsion beads are released using the same separation device (e.g. spin filter) using a release solution that breaks the interaction between the amplified bead and enrichment bead. For example, the spin filter with the emulsion beads attached to the captured enrichment beads is moved to a new tube (e.g. spin column). After the release solution is applied, the tube is centrifuged and the beads with amplicons are eluted and go to the bottom of the tube. The enrichment beads remain trapped in the filter. The beads with amplicons are collected and the filter with the trapped enrichment beads is discarded.

It is not intended that the present invention be limited to how the enriched live beads are subsequently used. In one embodiment, the amplicon on the enriched beads is sequenced. In one embodiment, the enriched beads are cross-linked to a flow cell for sequencing by synthesis.

It is not intended that the present invention be limited by how the enrichment beads capture the emulsion beads. In one embodiment, a portion of said emulsion beads of step e) comprise amplicon labeled with biotin and said enrichment beads comprise streptavidin-coated microspheres (or neutravidin-coated beads). It is not intended that the present invention be limited by the method by which amplicon becomes biotin labeled. In one embodiment, biotin is introduced into said amplicon during the amplification of step b) so as to create said amplicon labeled with biotin (e.g. by using one or more biotin-labeled primers). In one embodiment, for emulsion PCR, a biotinylated forward primer is on the bead and the reverse primer is in solution. In one embodiment, biotin-labeled oligonucleotides are hybridized to said amplicon after step c) so as to create said amplicon labeled with biotin.

When a single-strand specific exonuclease is applied to an enrichment protocol of live beads after emulsion PCR, the method resulted in significantly improved enrichment. Implementation of an Exonuclease I treatment step on GeneRad QlAcube does not require significant modification of the current instrument. Moreover, there are potential applications to other workflows and emulsion PCR live beads enrichment in general.

In one embodiment, the various methods and processes described above are automated. For example, the enriching method may be performed using an automated sample processing system. The system may have regions for particular tasks, e.g. centrifugation, to which and from which materials, e.g. tubes containing beads, are moved by a robotic arm or the like. The regions may have platforms, drawers, or decks. The commercially available QIAcube from Qiagen is equipped with an automated centrifuge and pipetting system which can be programmed to do all or a portion of the method steps with limited human intervention.

While not intending to be limited to any particular automated system or device, the system or device may comprise a deck, the deck comprising a plurality of sample carrier elements that may even be removably configured. The sample carriers may be both movable and removable as one piece or in pieces. The sample carriers may be positioned over a thermoblock, allowing for temperature cycling and amplification. This deck might be later removed and replaced with sample carriers positioned over a magnet, allowing for easy separation of magnetic particles, e.g. magnetic beads.

The sample processing control system may automate the sample processing system such that one or more tubes or plates (e.g. microtiter plate) may be processed according to one or more protocols. This sample processing may comprise one or more sampling protocols and steps, such as (but not limited to) adding reagents, mixing, centrifuging, removing supernatant, adding wash buffer, centrifuging again, removing supernatant, pipetting, and the like.

The automatic processing device may comprise a robotic arm having robotic movement, and in some embodiments, Cartesian movement. The arm may comprise one or more elements, such as a syringe, pipette or probe, a sensor element volume fluid and/or air applicator. The syringe, pipette or probe may be fluidically connected with a reservoir or other container, and may apply one or more of the following: rinse agents (e.g. buffers and the like), denaturing reagents (for separating DNA duplexes), additional materials (including beads). The syringe, pipette or probe may be fluidically connected to a vacuum or pump for the aspiration of reagents, such as aspiration of supernatant.

The sample processing system is configured to achieve an appropriate sequence of events that achieves a desired result to some degree. In achieving this sequence in an automated fashion to some degree the sample processing system is deemed an automated sample processing system and achieves automatic processing of at least one sample. This automated sequence as well as other aspects of the invention may be controlled by hardware, software, or some combination of them to accomplish a desired sequence with limited human intervention.

### DEFINITIONS

As used herein an “amplicon” is a product of an amplification reaction. An amplicon is typically double-stranded, but can be rendered single-stranded if desired. An amplicon corresponds to any suitable segment or the entire length of a nucleic acid target

As used herein, “particle” refers to discrete, small objects that may be in various shapes, such as a sphere (e.g. bead), capsule, polyhedron, and the like. Particles can be macroscopic or microscopic, such as microparticles or nanoparticles. Particles can be non-magnetic or magnetic. Magnetic particles may comprise a ferromagnetic substance, and the ferromagnetic substance may be Fe, Ni, Co, an iron oxide or the like.

The “beads” used herein may be fabricated from any number of known materials. Example of such materials include: inorganics, natural polymers, and synthetic polymers. Specific examples of these materials include: cellulose, cellulose derivatives, acrylic resins, glass, silica gels, polystyrene, gelatin, polyvinyl pyrrolidone, co-polymers of vinyl and acrylamide, polystyrene, polyacrylamides, latex gels, dextran, rubber, silicon, plastics, nitrocellulose, natural sponges, silica gels, control pore glass, metals, cross-linked dextrans (e.g., Sephadex™), agarose gel (Sepharose™), and other solid phase supports known to those of skill in the art. In preferred embodiments, the emulsion beads are beads approximately 1 micron in diameter.

For use with the present invention, emulsion beads with or without attached nucleic acid template are suspended in a heat stable water-in-oil emulsion. It is contemplated that a portion of the microdroplet population include only one template and one bead. There may be many droplets that do not contain a template or which do not contain a bead. Likewise there may be droplets that contain more than one copy of a template. The emulsion may be formed according to any suitable method known in the art. One method of creating emulsion is described below but any method for making an emulsion may be used. These methods are known in the art and include adjuvant methods, counter-flow methods, cross-current methods, rotating drum methods, and membrane methods. Furthermore, the size of the microcapsules may be adjusted by varying the flow rate and speed of the components. For example, in dropwise addition, the size of the drops and the total time of delivery may be varied. Preferably, the emulsion contains a density of between about 10,000-1,000,000 beads encapsulated per microliter. This number depends on the size of the microspheres, droplets and the ratio of emulsion phases (i.e., oil to aqueous).

As described herein, after amplification the emulsion is “broken” (also referred to as “de-emulsification” in the art). There are many methods of breaking an emulsion. Processes for breaking emulsions known in the prior art include processes that use an inorganic or organic de-emulsifier, and processes that treat emulsions mechanically. One preferred method of breaking the emulsion uses additional oil to cause the emulsion to separate into two phases. The oil phase is then removed, and a suitable organic solvent is added. After mixing, the oil/organic solvent phase is removed. This step may be repeated several times. Finally, the aqueous layers above the beads are removed. The beads are then washed with a mixture of an organic solvent and annealing buffer (e.g., one suitable hybridization buffer or “annealing buffer” is described in the examples below), and then washed again in annealing buffer. Suitable organic solvents include alcohols such as methanol, ethanol, isopropanol and the like. In another embodiment the emulsion is broken by the addition of organic phase that solubilizes both aqueous phase and the oil/detergent and the homogenous solution removed after centrifugation or magnetic separation. The workup is usually then followed by washes with aqueous buffers, such as PBS with additional detergent (Tween-20).

### DESCRIPTION OF THE INVENTION

Methods and compositions for improving the enrichment of a population of particles containing an analyte are disclosed. The technique finds many uses, including but not limited to enriching for emulsion beads with clonally PCR amplified template (“live beads”), enriching for beads with desired DNA/RNA sequences, and capture of specific DNA and RNA targets with microspheres.

In one embodiment, the present invention contemplates a method for improving the enrichment of clonally amplified nucleic acid by employing a nuclease such as an endonuclease or an exonuclease. In one embodiment, the present invention contemplates us of an exonuclease, such as E. coli Exonuclease I, to increase the specificity of affinity-based isolations of nucleic acid-containing microspheres, and to decrease non-specific bead-to-bead interactions of nucleic acid-containing microspheres. E. coli Exonulcease I is a highly processive enzyme catalysing the removal of nucleotides from single-stranded DNA in the 3′ to 5′ direction. Thereby, single-stranded DNA fragments (for example PCR primers) present either in solution or bound to microspheres, which may lead to unspecific interactions, are specifically degraded, while double-stranded DNA-DNA hybrids mediating the interaction and isolation are unaffected.

### EXPERIMENTAL

We clonally amplified NGS-libraries by solid-phase emulsion PCR on primer-conjugated microspheres (MyOne streptavidin coated magnetic beads purchased from LifeTech saturated with bisbiotinylated forwardprimer). Briefly, beads, PCR components and a limited dilution of template were mixed with an oil phase and emulsified on GeneRead QiaCube in order to generate PCR microcompartments (emulsions). The emulsions were then subjected to PCR. After removal of all oil phase compartments following PCR, approximately 10% of the microspheres contained template DNA. To facilitate the isolation of template-containing microspheres, biotin-labelled oligonucleotides specific for amplicons generated during emulsion PCR (added either during emulsion PCR or by hybridization) were used.

Next, an enrichment experiment was performed where microspheres with biotin-labelled amplicons were isolated using streptavidin-coated polystyrene beads. The effect of Exonuclease I was tested by pre-treating the microspheres generated during emulsion PCR with 2 U/ul Exonuclease I (New England Biolabs, Cat. No. M0293L) in Exonuclease buffer, or Exonuclease buffer only, using the following conditions:
Beads (after removal of solution/supernatant on magnetic stand)

### 10 ul 10×Exol Reaction Buffer (NEB)

### 10 ul Exonuclease I (20 U/μl)

### 80 ul H₂O

Incubation conditions: 1 hour at 37° C.
   As shown in Table 1, the treatment of Exonuclease I significantly improved the specificity of the enrichment of amplicon harboring microspheres. Live beads were detected by FACS analysis for the data in Table 1.

TABLE 1
Average of 8/4 enrichment experiments using the same material
(microspheres after emulsion PCR). The treatment with Exonuclease I
significantly increases the specificity of the the binding of live beads
to capture beads, thereby leading to higher percentage of live beads.

| | Number | | % live beads in | % live beads |
|---|---|---|---|---|
| | replicates | Treatment | starting material | after enrichment |
| Bio/Strep T28 | 8 | | 6.4 | 24.3 |
| Bio/Strep T28 | 4 | Exonuclease I | | 46.5 |

1. A method of recovering amplified nucleic acid, comprising:
a) providing i) a plurality of amplification beads, amplification reagents, a first primer immobilized on said beads, a second primer in solution, and template; ii) enrichment beads, wherein said enrichment beads are different from said amplification beads, and iii) a single-strand specific nuclease;
b) exposing said amplification beads to conditions so as to amplify at least some of said template on at least some of said beads so as to create processed beads;
c) treating said processed beads with said single-strand specific nuclease so as to create treated beads; and
d) contacting said treated beads with said enrichment beads, wherein said treated beads comprising amplified template bind to said enrichment beads so as to make a population of bead complexes, thereby recovering amplified nucleic acid.

2. The method of claim 1, wherein said treated beads not comprising amplified template do not bind to said enrichment beads in step d).

3. The method of claim 1, wherein a portion of said treated beads of step d) comprise amplicon labeled with biotin and said enrichment beads comprise streptavidin-coated microspheres.

4. The method of claim 3, wherein biotin was introduced into said amplicon during amplification of step b) so as to create said amplicon labeled with biotin.

5. The method of claim 3, wherein biotin-labeled oligonucleotides were hybridized to said amplicon after step c) so as to create said amplicon labeled with biotin.

6. The method of claim 1, wherein said amplification reagents comprise PCR reagents.

7. A method of enriching, comprising:
a) providing i) an emulsion comprising one or more aqueous compartments in oil, at least some of said compartments comprising PCR reagents, a first primer immobilized on an emulsion bead, a second primer in solution, and template; ii) enrichment beads, wherein said enrichment beads are different from said emulsion beads in said compartments, and iii) a single-strand specific nuclease;
b) exposing said emulsion to conditions so as to amplify at least some of said template on at least some of said emulsion beads in at least some of said compartments;
c) breaking said emulsion under conditions such that said emulsion beads are recovered;
d) treating said recovered emulsion beads with said single-strand specific nuclease to as to create treated beads; and
e) enriching for treated beads comprising amplified template by contacting said treated beads with said enrichment beads, wherein said treated beads comprising amplified template bind to said enrichment beads so as to make a population of treated bead—enrichment bead complexes.

8. The method of claim 7, wherein said treated beads not comprising amplified template do not bind to said enrichment beads in step e).

9. The method of claim 7, wherein a portion of said treated beads of step e) comprise amplicon labeled with biotin and said enrichment beads comprise streptavidin-coated microspheres.

10. The method of claim 9, wherein biotin was introduced into said amplicon during amplification of step b) so as to create said amplicon labeled with biotin.

11. The method of claim 9, wherein biotin-labeled oligonucleotides were hybridized to said amplicon after step c) so as to create said amplicon labeled with biotin.

12. The method of claim 7, further comprising f) capturing at least some of said population of complexes under conditions such that a majority of said treated beads not comprising amplified template are not captured.

13. The method of claim 12, wherein the capturing in step f) comprises size selection.

14. The method of claim 13, wherein said size selection comprises density centrifugation.

15. The method of claim 13, wherein said size selection comprises capturing at least some of said population of complexes on a surface.

16. The method of claim 15, wherein said surface comprises the surface of a filter.

17. The method of claim 16, wherein said filter is a single layer nylon mesh.

18. The method of claim 16, wherein said filter is positioned in a spin column.

19. The method of claim 18, wherein said spin column is centrifuged during step f) so as to facilitate passage of said uncaptured beads through said filter.

20. The method of claim 7, wherein said enrichment beads are different in size from said emulsion beads.

21. The method of claim 20, wherein said enrichment beads are at least five times and up to one hundred times larger than said emulsion beads.

22. The method of claim 12, further comprising step g): subjecting said population of complexes to conditions so as to separate said treated beads comprising amplified template from said enrichment beads such that the majority of said treated beads comprising amplified template separate from said enrichment beads.
